(19) United States
(12) Patent Application Publication
(10) Pub. No.: US 2007/0179166 A1
(43) Pub. Date: Aug. 2, 2007

(54) Process for preparation of statins with high syn to anti ratio

(76) Inventors: Valerie Niddam-Hildesheim, Kadima (IL); Anna Balanov, Rehovot (IL); Kobi Chen, Kfar-Saba (IL)

Correspondence Address: KENYON & KENYON LLP, ONE BROADWAY, NEW YORK, NY, 10004, US

(21) Appl. No.: 11/520,295

(22) Filed: Sep. 12, 2006

Related U.S. Application Data
(63) Continuation-in-part of application No. 11/020,834, filed on Dec. 23, 2004.
(60) Provisional application No. 60/716,802, filed on Sep. 12, 2005.
(60) Provisional application No. 60/532,458, filed on Dec. 24, 2003.
(60) Provisional application No. 60/547,715, filed on Feb. 24, 2004.

(51) Int. Cl. A61K 31/495; C07D 239/24

(52) U.S. Cl. 514/275; 544/332

(57) ABSTRACT

Provided is a process for reduction of statin ketoesters and purification of diol esters of the statins through selective crystallization.

### CROSS REFERENCE TO RELATED APPLICATIONS

This application claims the benefit of U.S. Provisional Application Ser. No. 60/716,802, filed Sep. 12, 2005, and is a continuation-in-part of U.S. application Ser. No. 11/020,834, filed Dec. 23, 2004, which claims the benefit of U.S. Provisional Application Ser. Nos. 60/532,458, filed Dec. 24, 2003 and 60/547,715, filed Feb. 24, 2004, the disclosures of all of which are incorporated by reference in their entirety herein.

### FIELD OF THE INVENTION

The present invention related to reduction of statins and increasing their syn to anti ratio. In particular, the present invention provides methods of reducing a ketoester intermediate of rosuvastatin.

### BACKGROUND OF THE INVENTION

The class of drugs called statins are currently the most therapeutically effective drugs available for reducing low-density lipoprotein (LDL) particle concentration in the blood stream of patients at risk for cardiovascular disease and thus, statins are used in the treatment of hypercholesterolemia, hyperlipoproteinemia, and atherosclerosis. A high level of LDL in the bloodstream has been linked to the formation of coronary lesions that obstruct the flow of blood and can rupture and promote thrombosis. Goodman and Gilman, The Pharmacological Basis of Therapeutics, page 879 (9th Ed. 1996).

Statins inhibit cholesterol biosynthesis in humans by competitively inhibiting the 3-hydroxy-3-methyl-glutaryl-coenzyme A (“HMG-CoA”) reductase enzyme. HMG-CoA reductase catalyzes the conversion of HMG to mevalonate, which is the rate determining step in the biosynthesis of cholesterol. Decreased production of cholesterol causes an increase in the number of LDL receptors and corresponding reduction in the concentration of LDL particles in the bloodstream. Reduction in the LDL level in the bloodstream reduces the risk of coronary artery disease. J.A.M.A. 1984, 251, 351-74.

Currently available statins include lovastatin, simvastatin, pravastatin, fluvastatin, cerivastatin and atorvastatin. Lovastatin (disclosed in U.S. Pat. No. 4,231,938) and simvastatin (ZOCOR; disclosed in U.S. Pat. No. 4,444,784 and WO 00/53566) are administered in the lactone form. After absorption, the lactone ring is opened in the liver by chemical or enzymatic hydrolysis, and the active hydroxy acid is generated. Pravastatin (PRAVACHOL; disclosed in U.S. Pat. No. 4,346,227) is administered as the sodium salt. Fluvastatin (LESCOL; disclosed in U.S. Pat. No. 4,739,073) and cerivastatin (disclosed in U.S. Pat. Nos. 5,006,530 and 5,177,080), also administered as the sodium salt, are entirely synthetic compounds that are in part structurally distinct from the fungal derivatives of this class that contain a hexahydronaphthalene ring. Atorvastatin and two new “superstatins,” rosuvastatin and pitavastatin, are administered as calcium salts. The structural formulas of these statins are shown below.

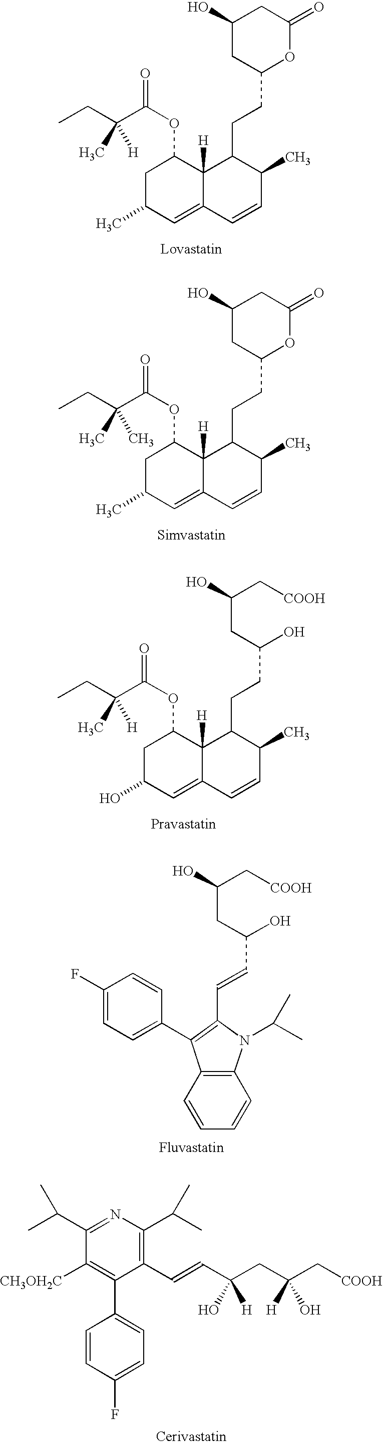

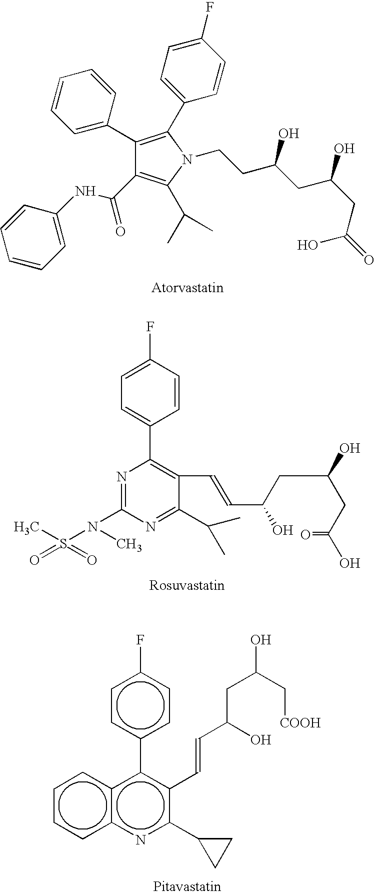

[R*,S*-(E)]-(±)-7-[3-(4-fluorophenyl)-1-(1-methylethyl)-1H-indol-2-yl]-3,5-dihydroxy-6-heptenoic acid is fluvastatin and its structure is depicted above.

A step in the synthesis of statins is reduction of a ketoester to yield the statin. For example, with fluvastatin, in U.S. Pat. No. 5,354,772, a ketoester of fluvastatin is reduced with EtB₃/NaBH₄ to obtain a diol ester. In another patent, U.S. Pat. No. 5,189,164 (EP 0 363 934), a ketoester of fluvastatin is reduced with diethylmethoxyborane to provide fluvastatin. Both these US patents relate to a process of purifying the FLV-diol ester by chromatography only. In U.S. Pat. No. 5,260,440, relating to rosuvastatin and in the U.S. Pat. No. 5,856,336, relating to pitavastatin, the statin-diol esters are also isolated by chromatography. In example 8 of WO 03/004455, 6-dibenzylcarbamoyl-5-hydroxy-3-oxo-hexanoic acid tert-butyl ester is reduced by hydrogenation at a pressure of 25 bar, followed by drying of ethyl acetate to obtain a residue having a syn to anti ratio of 7.6 to 1.

Reduction of a ketoester is also disclosed in Tetrahedron 49, 1997-2010 (1993). In the paper, reduction of a ketoester, which is not a particular statin, is carried out by EtB₃/NaBH₄ or RU-binap to provide a diol ester. In another paper, a ketoester, which is also not any particular statin, is reduced by catecholborane in the optional presence of Rh(PPh₃)Cl. JOC 55, 5190-5192 (1990).

The choice of reducing agents is an important factor in obtaining a statin from its corresponding ketoester since it influences the ratio of syn to anti obtained. The United States Pharmacopeia (USP) provides standards regarding the ratio of syn to anti that is used in a statin formulation. The USP requirements dictate use of a reducing agent that allows obtaining a high syn to anti ratio.

There is a need in the art for reducing agents which may be employed on an industrial scale on a cost effective basis, and which provide a high ratio of syn to anti and overall yield.

The diol ester obtained after reduction is usually not isolated, and is hydrolyzed to obtain a salt. For example, in U.S. Pat. No. 5,003,080, the intermediate ester isn't isolated at all. In one instance however, in Journal of Labeled Compounds & Radiopharmaceuticals vol. XLI, pages 1-7 (1988), a fluvastatin diol ester is obtained from hexane containing 3% isopropanol by volume. (See also TETRAHEDRON, VOL. 53 (31), 10659-10670, 1997)

We have yet found additional ways to increase the Syn to anti ratio of statins through isolation of the diol ester.

### SUMMARY OF THE INVENTION

In one embodiment, the present invention provides a process for preparing rosuvastatin diol ester comprising the steps of

a) combining B-Methoxy-9-BBN, an organic and a ketoester having the formula:

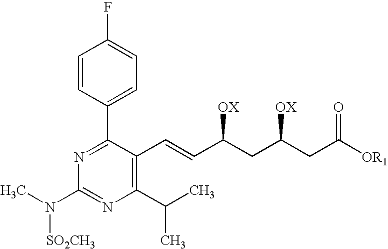

wherein R₁ is a straight or branched C₁ to C₄ alkyl group, and wherein at least one X forms a double bond to give a ketone, and at most one X is a hydrogen, to obtain a reaction mixture,

b) combining a source of hydride ions with the reaction mixture, and

c) maintaining the reaction mixture to obtain the rosuvastatin diol ester.

In another embodiment, the present invention provides a process for preparing rosuvastatin from a rosuvastatin diol-ester having the formula:

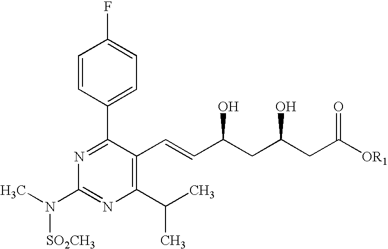

wherein R₁ is a straight or branched C₁ to C₄ alkyl group; comprising the steps of

a) combining a ketoester of rosuvastatin having the formula:

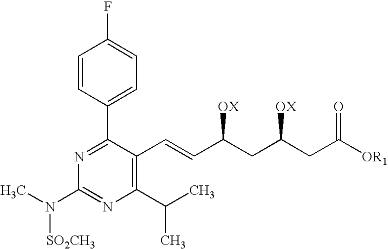

with a solvent to form a solution, wherein R₁ is a straight or branched C₁ to C₄ alkyl group, and wherein at least one X forms a double bond to give a ketone, and at most one X is a hydrogen;

b) cooling the solution to a temperature of about −50° C. to about −80° C.;

c) combining B-Methoxy-9-BBN with the solution to obtain a reaction mixture, and maintaining the reaction mixture for at least about 30 minutes;

d) combining a source of hydride ions with the reaction mixture, and maintaining the reaction mixture for an additional period of at least about 2 hours;

e) quenching the reaction mixture;

f) recovering the rosuvastatin diol-ester; and

g) converting the rosuvastatin diol-ester to rosuvastatin or a pharmaceutically acceptable salt of rosuvastatin.

In another embodiment, the present invention provides a process for preparing rosuvastatin from a rosuvastatin ketoester having the formula: 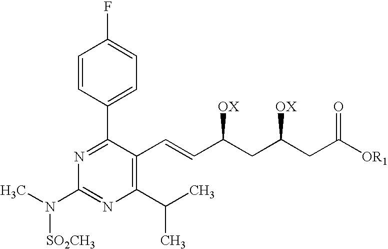
wherein R₁ is a straight or branched C₁ to C₄ alkyl group, and wherein at least one X forms a double bond to give a ketone, and at most one X is a hydrogen, comprising the steps of

a) combining the ketoester of rosuvastatin with a solvent to form a solution;

b) cooling the solution to a temperature of about −50° C. to about −80° C.;

c) combining B-Methoxy-9-BBN with the solution to obtain a reaction mixture and maintaining the reaction mixture for at least about 30 minutes;

d) combining a source of the hydride ions to the reaction mixture and maintaining the reaction mixture for an additional period of at least about 2 hours to obtain rosuvastatin diol ester;

e) quenching the reaction mixture;

f) combining the rosuvastatin diol ester with NaOH or Ca(OH)₂ and a solvent or a mixture of solvent and water; and

g) recovering the rosuvastatin free acid, lactone or a pharmaceutically acceptable salt thereof.

### DETAILED DESCRIPTION OF THE INVENTION

The present invention provides methods for reduction of a statin ketoester by use of 9-methoxy-9-bora-bicyclo[3.3.1]nonane (B-methoxy-9-BBN) as a complexant agent. Complexation with B-methoxy-9-BBN (BM-9-BBN) provides ideal selectivity. The requirement for fluvastatin diol ester is no more than about 0.8% by area % HPLC of the anti product. The reduction process of the present invention yields, in case of fluvastatin, about 0.5 to 0.6% anti by area % HPLC, and other crystallization steps yield less than about 0.2% anti by area % HPLC. Additionally, B-methoxy-9-BBN may be used in a molar ratio as low as about 1:1.

The ketoester reduced in the present invention, which is exemplified by fluvastatin and rosuvastatin, has the following formula: 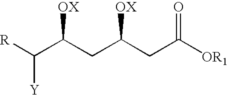
wherein R₁ is a C₁ to C₄ alkyl group (t-butyl preferred), R is an organic radical as described below, Y is a hydrogen or forms a double bond with the R group and at least one of the X's forms a double bond with the carbons being attached to the oxygen to give a ketone, and at most one X is hydrogen. A preferred reaction scheme is illustrated below, where the X closest to the ester forms a ketone and the other X is a hydrogen (alpha ketoester):

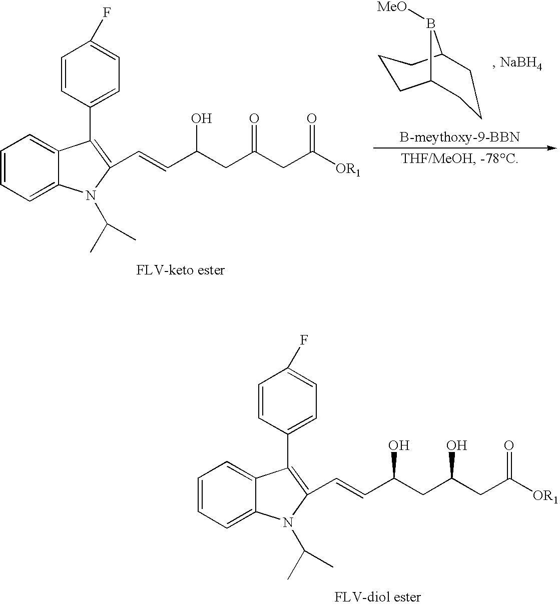

As used herein, R₁ refers to an organic radical that is bonded to the diol pentanoic ester group and is inert to reduction with the reducing agent and allows for therapeutic activity. By inert to reduction it is meant that the reducing agent employed does not reduce the R Group according to the general knowledge of one of skill in the art. Depending on the statin, the R radical can be:
pravastatin: 1,2,6,7,8,8a-Hexahydro-6-hydroxy-2-methyl-8-(2-methyl-1-oxobutoxy)-1-naphthalene ethyl radical.
fluvastatin: 3-(4-fluorophenyl)-1-(1-methylethyl)-1H-indol-2-yl]-ethylene radical.
cerivastatin: 4-(4-fluorophenyl)-5-methoxymethyl)-2,6-bis(1-methylethyl)-3-pyridinyl-ethylene radical.
atorvastatin: 2-(4-fluorophenyl)-5-(1-methylethyl)-3-phenyl-4-[(phenylamino)carbonyl]-1H-pyrrole-ethyl radical.
rosuvastatin: [4-(4-fluorophenyl)-6-(1-methylethyl)-2-[methyl(methylsulfonyl)amino]-5-pyrimidinyl]-ethylene radical.
pitavastatin: [4′-(4″-fluorophenyl)-2′-cyclopropyl-quinolin-3′-yl]-ethylene radical.

The R radical can also be that of the open ring form, i.e., the dihydroxy acid, of simvastatin or lovastatin. These open ring forms also have a diol pentanoic acid group. As used herein, the terms simvastatin and lovastatin include both the lactone form and the open-ring form, unless otherwise indicated by a formula. When the statin is simvastatin or lovastatin, the R radical is:
simvastatin: 1,2,6,7,8,8a-Hexahydro-2,6-dimethyl-8-(2,2-dimethyl-1-oxobutoxy)-1-naphthalene ethyl radical.
lovastatin: 1,2,6,7,8,8a-Hexahydro-2,6-dimethyl-1-8-(2-methyl-1-oxobutoxy)-1-naphthalene ethyl radical.

The reduction of the statin ketoester, with B-Methoxy-9-BBN includes combining the statin ketoester and a solvent; cooling the solution to a temperature of about −50° C. to about −80° C.; adding B-Methoxy-9-BBN and maintaining the reaction mixture for at least about 30 minutes; adding a source of hydride ions and maintaining the reaction mixture for an additional period of at least about 2 hours; adding a quenching agent; and recovering the statin diol-ester. The solvent may include C, to C₄ alcohols such as methanol, dipolar solvents such as tetrahydrofuran, C₂ to C₈ ethers cyclic or acyclic, or a mixture thereof. Preferably, the solution is cooled to about −70° C. to about −80° C. An optimum temperature is about −70° C., which allows for greater selectivity. The source of hydride ions may be sodium borohydride, potassium borohydride and lithium borohydride, preferably sodium borohydride. The quenching agent may be any one of hydrogen peroxide, sodium carbonate·1.5H₂O or NaBO₃·H₂O, 3-chloroperbenzoic acid, ammonium chloride, aqueous solution of HCl, acetic acid, oxone, sodium hypochlorite, dimethyl disulfide, diethanolamine, hydroxylamine-O-sulfonic acid, acetone, preferably hydrogen peroxide. The quenching agent is used for terminating the reaction, by reacting it with the remaining reducing agent.

After quenching the reaction, the statin diol-ester may be recovered from the reaction mixture by adding a C₄ to C₇ ester and water, separating the organic phase from the two-phase system that formed, and removing the solvent by any technique known in the art (such as evaporation).

In one embodiment of the invention, the process of preparing a C₁ to C₄ alkyl ester of rosuvastatin, preferably t-butyl rosuvastatin ester (TBRE), includes adding a source of hydride ions to a solution of the rosuvastatin ester and MeO-9-BBN. This process includes forming a complex of the keto-ester and MeO-9-BBN, followed by reduction with a source of hydride ions.

In a preferred embodiment, the process includes the steps of: providing a solution of rosuvastatin C₁-C₄ keto-ester and MeO-9-BBN in an organic solvent; adding a source of hydride ions to the solution; and maintaining the solution for a time sufficient to obtain the corresponding diol ester.

In one embodiment, the C₁-C₄ ester, including TBRE has diastereomeric impurities of 0.37%.

The solution of rosuvastatin keto-ester and MeO-9-BBN may be prepared by combining the rosuvastatin keto-ester and MeO-9-BBN with a suitable organic solvent. Preferably, a dilution of MeO-9-BBN of about 30 to about 60 volumes (vs. rosuvastatin keto-ester) is used in the process of the invention.

Suitable organic solvents include C₁ to C₄ alcohols, polar solvents, cyclic or acyclic C₃ to C₈ ethers, and mixtures of these. Specific examples of solvents include methylene chloride, toluene, methyl t-butyl ether, di-ethyl ether, tetrahydrofuran, dioxane, methanol, ethanol, isopropanol and n-butanol.

In one preferred embodiment, the reduction of rosuvastatin ketoester to rosuvastatin diol ester may be carried out in a mixture of methanol and THF. Other solvents as specified above may be used. The optimum temperature of the reduction is at a temperature below about −70° C., more preferably about −78° C. B-Methoxy-9-BBN is added to a solution of the ester at this optimal temperature, followed by addition of a suitable source of hydride ions. The source of hydride ions may be sodium borohydride, potassium borohydride and lithium borohydride, preferably sodium borohydride. Sodium borohydride is a preferred source of hydride ions in this embodiment of the invention.

The reaction may be quenched after its completion. Preferably, the quenching agent is selected from the group consisting of: hydrogen peroxide, 3-chloroperbenzoic acid, ammonium chloride, aqueous solution of HCl, acetic acid, oxone, sodium hypochlorite, dimethyl disulfide, diethanolamine, hydroxylamine-O-sulfonic acid and acetone. More preferably, the quenching agent is hydrogen peroxide.

Rosuvastatin diol ester may be recovered from a biphasic mixture of water and a water immiscible organic solvent, where the ester moves into the organic phase. It may then be washed under basic and brine conditions. Preferred water immiscible organic solvents are ethyl acetate, toluene or methyl ethyl ketone, with ethyl acetate being most preferred.

According to USP pharmacopoeia, the level of anti-isomer should be NMT 0.8% (% area by HPLC according to USP HPLC method). In order to increase the syn to anti isomer ratio the statin diol-ester may be crystallized.

In one embodiment, fluvastatin diol-ester in the present invention may be crystallized from the following solvents: C₃ to C₇ ketone such as acetone, C₁ to C₄ alcohol such as ethanol, isopropyl alcohol, 1-propanol, 2-propanol 1-butanol and 2-butanol, C₃ to C₇ ester other than ethyl acetate such as isopropylacetate, isobutylacetate or methyl acetate, C₁-C₄ ethers other than MTBE (methyl t-butyl ether), and mixtures thereof. The crystallization solvent may also be a mixture of MTBE and C₁ to C₄ alcohols, preferably MTBE and IPA (iso-propanol). The crystallization includes the steps of: dissolving the statin diol-ester in said solvent at elevated temperature; cooling the solution; and recovering the crystallized fluvastatin diol ester. Preferably, the solvent is selected from the group consisting of: acetone, IPA, isopropylacetate, acetonitrile, mixtures thereof (with or without water) and a mixture of IPA/MTBE. The elevated temperature is preferably above about 30° C., more preferably above about 40° C. and most preferably about reflux temperature.

The precipitate obtained may be recovered by conventional techniques such as filtration and concentration. Preferably, the fluvastatin is dissolved at reflux. Seeding may also be used for crystallization.

The fluvastatin diol-ester may also be crystallized by using a solvent and an anti-solvent. This comprises the steps of: dissolving the statin diol-ester in a C₃ to C₇ ketone solvent such as acetone, methylethylketone and methyl isopropyl ketone, at elevated temperature; adding a C₅ to C₁₂ saturated hydrocarbon such as cyclic and acyclic heptane and hexane; cooling the solution; and recovering the crystallized diol ester. Preferably, the cooling is at a temperature of from about 10° C. to about 25° C. Preferably, the elevated temperature is the reflux temperature. In one embodiment, a C₁ to C₄ alcohol is used with less than 50% hydrocarbon by volume, more preferably without a hydrocarbon.

The term “anti-solvent” refers to a liquid that, when added to a solution of fluvastatin diol ester in a solvent, induces precipitation of fluvastatin sodium. The anti-solvent may also be in a binary mixture with the solvent when the solution is prepared. Precipitation of fluvastatin diol ester is induced by the anti-solvent when addition of the anti-solvent causes fluvastatin diol ester to precipitate from the solution more rapidly or to a greater extent than fluvastatin diol ester precipitates from a solution containing an equal concentration of fluvastatin diol ester in the same solvent when the solution is maintained under the same conditions for the same period of time but without adding the anti-solvent. Precipitation can be perceived visually as a clouding of the solution or formation of distinct particles of fluvastatin diol ester suspended in or on the surface of the solution or collected on the walls or at the bottom of the vessel containing the solution.

The above crystallizations may allow for increasing the syn to anti ratio so that the level of the anti isomer is about 0.2 or less % area by HPLC. Preferably the level of the anti isomer is about 0.04 or less % area by HPLC.

In another embodiment, rosuvastatin diol ester is crystallized or slurried. Crystallization of the diol ester includes preparing a solution of the C1-C4 ester, including TBRE in a solvent selected from the group consisting of: C₁-C₄ alcohols, C₃-C₈ esters, C₃-C₈ ketones, C₃-C₈ ethers, C₆ to C₁₀ aromatic hydrocarbons, PGME (propylene glycol monomethyl ether), water, acetonitrile, and mixtures thereof; cooling the solution to crystallize the diol ester; and recovering the crystallized diol ester. Slurrying can be carried out in the same solvents, followed by recovery of the diol ester. Preferably, the recovery comprises filtering the slurry to obtain a precipitate. Preferably, the filtration is under reduced pressure. Preferably, the obtained precipitate is further dried.

Most preferably the solvent of crystallization is toluene or a mixture of methanol/water or acetonitrile/water.

The diol ester may be further converted into a pharmaceutically acceptable salt of the statin or a lactone. In one embodiment, the diol ester obtained is reacted with sodium or calcium hydroxide to obtain the sodium or calcium salt. It is also possible to first obtain the sodium salt by reaction with sodium hydroxide, and then convert the sodium salt to calcium salt by using a source of calcium such as calcium chloride or calcium acetate. The basic hydrolysis of the statin diol-ester may be carried out with one or more equivalents of an alkali metal or alkaline earth metal base such as NaOH or Ca(OH)₂, in organic solvents such as C₁ to C₈ ethers (tetrahydrofuran, IPE), acetonitrile (ACN), C₁ to C₄ alcohols (MeOH, EtOH, IPA,, propanol, butanol etc.), C₃ to C₈ ketones or esters (acetone, methyl ethyl ketone, methyl isopropyl ketone, ethyl acetate). The hydrolysis may also be carried out with water, a mixture of the above solvents, or a mixture of water and the above solvents, preferably at room temperature or by heating. The lactone, particularly for fluvastatin, may be obtained by treating the acid form with an acid such as HCl.

Pharmaceutical Compositions

Pharmaceutical formulations of the present invention contain pharmaceutically acceptable salts or lactone form of the statin with a high syn to anti ratio. Pharmaceutically acceptable salts include those of alkali and alkaline earth metals, preferably calcium. In addition to the active ingredient(s), the pharmaceutical compositions of the present invention may contain one or more excipients or adjuvants. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, statin and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets and troches, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compac ted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

Having thus described the invention with reference to particular preferred embodiments and illustrated it with Examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications.

HPLC Method for Diastereomer Content in Tert-Butyl Ester of Rosuvastatin

HPLC conditions:

| | |
|---|---|
| Column | BDS Hypersil C18 |
| Mobile phase | Gradient of Buffer and Organic modifier |
| Buffer | Ammonium acetate buffer |
| Organic modifier | Acetonitrile and Ethanol |
| Detection | UV-245 nm |
| Injection | 10 μl |
| Column temperature | 5° C. |
| Diluent | Acetonitrile/Water |

Sample preparation:

0.5mg/ml in diluent
Calculations:

$$\% 3 R , 5 R - isomer = \frac{Area 3 R , 5 R - isomer in smp . \times 100 \%}{\sum all Areas}$$

HPLC Method for Diastereomer Content in Rosuvastatin Ca

HPLC Conditions:

| | |
|---|---|
| Column | C18 |
| Mobile phase | Gradient of Buffer and Organic modifier |
| Buffer | Ammonium acetate buffer |
| Organic modifier | Acetonitrile and Ethanol |
| Detection | UV-243 nm |
| Injection | 10□l |
| Column temperature | 20° C. |
| Diluent | Acetonitrile/Buffer |

Sample preparation:

0.2mg/ml in diluent
Calculations:

$$\% 3 R , 5 R - isomer = \frac{Area 3 R , 5 R - isomer in smp . \times 100 \%}{\sum all Areas}$$

### EXAMPLES

### Example 1

### Reduction of FKE-tBu to FDE-tBu

A IL triple-jacket reactor, covered with aluminum foil was loaded with FKE-tBu (30 g), THF (Tetrahydrofuran) (CP, 300 ml) and Methanol (CP, 60 ml). The solution was cooled to (−70° C.) and then BM-9-BBN (IM solution in Hexanes, 71 ml.) was added. The mixture was stirred at (−70° C.) for 30 minutes. Sodium borohydride (2.4 g) was added and the reaction mixture was stirred at (−70° C.) for about 2 hours (monitoring by HPLC for the consumption of FKE-tBu). A solution of 30% Hydrogen peroxide (48 ml) was added and the reaction mixture was allowed to stir at room temperature for 19.5 hours. The reaction mixture was diluted with EtOAc (ethyl acetate) (150 ml), water (150 ml) and Brine (105 ml). The phases were separated and the organic layer was washed with saturated solution of NaHCO₃ (1×120 ml), saturated solution of Na₂SO₃ (1×120 ml) and Brine (1×120 ml). The organic layer was evaporated under vacuum to dryness.

The obtained solid residue was dissolved in acetone (90 ml) at reflux temperature while the flask was covered with aluminum foil. Then n-Heptane (210 ml) was added at reflux. The mixture was cooled to room temperature and stirred at this temperature for about 18 hours. The product was isolated by filtration under nitrogen atmosphere, washed with n-Heptane (100 ml) and dried at 40° C. in a vacuum oven for 24 hours to obtain 21.9 g (73%) of FDE-tBu crude. First crystallization-Syn:anti-99.0/0.45.

### Example 2

### Crystallization of Crude FLV-diol Ester from Acetone and n-Heptane

FDE-tBu crude (syn:anti 99.0:0.45) was dissolved in Acetone (116 ml) at reflux temperature while the flask was covered with aluminum foil. Then n-Heptane (252 ml) was added at reflux. The mixture was cooled to 37° C. during 1 hour, stirred at this temperature for 1 hour and cooled to 20° C. during 1 hour. The obtained slurry was stirred at 20° C. for 15 hours. The product was isolated by filtration under nitrogen atmosphere, washed with n-Heptane (3×66 ml) and dried at 40° C. in a vacuum oven for 24 hours to obtain 18.9 g (90%) of FDE-tBu cryst (syn:anti 99.8:0.17).

### Example 3

### Conversion of FDE-tBu to FLV Na Form XIV

Water (56 ml), ACN (Acetonitrile) (200 ml) and FDE-tBu (40 gr) are added to a 1 L stirred reactor. At 25 deg. 7.5 gr of 47% NaOH solution are added and the mixture is heated to 35° C. The mixture becomes clear during the hydrolysis. End of reaction is determined by HPLC (˜3-4 hr). The mixture is then cooled to 25° C. ACN (Acetonitrile) (600 ml) is added to the mixture causing precipitation of FLV Na crystals. The mixture is stirred for ˜5 hr and then filtered under vacuum. The wet product is washed with 120 ml of ACN (Acetonitrile). The wet product is dried in a vacuum oven at 40° C. to obtain FLV Na form XIV crystals. Yield: 87 %

### Example 4

### Conversion of FDE-Me to FLV Na

Fluvastatin-diol methyl ester (3.0g) was added to solution of NaOH (1 eq.) in water (0.75 ml) and ethanol (7.5 ml). The mixture was heated to reflux and stirred until the raw material wasn't observed by HPLC. After this time 58 ml of MTBE were dripped to the solution during 1.5 hr. Turbidity appeared in the solution, which was cooled slowly to room temperature and stirred over night. The product was isolated by filtration under nitrogen, washed with MTBE (50 ml) and dried at 50° C. in a vacuum oven for 24 hours to obtain 2.21 grams (72.3%) of fluvastatin sodium.

### Example 5

### Conversion of FDE-ME to FLV Na

Fluvastatin-diol-methyl ester (FDE-ME) (4.0 g) was dissolved in acetone (40 ml). A solution of NaOH (0.38gr) in MeOH (4 ml) was added and the mixture was stirred at room temperature for 20 hr. The product was isolated by filtration under nitrogen, washed with acetone (20 ml) and dried at 50° C. in a vacuum oven for 26 hours to obtain 3.35 gr (82.2%) of fluvastatin sodium.

### Example 6

### Crystallization of Crude FLV-diol Ester From IPA

Crude FLV-diol-tert butyl ester (that prepared as mentioned in the reduction procedure with BM-9-BBM) (5.77 gr, Syn:anti-98.6/0.88) was dissolved in IPA (60 ml) by heating to reflux. After 30 minutes, the clear solution was cooled to room temperature and stirred over night. The solution was then concentrated (approximately 17 ml of IPA was evaporated) and stirred at room temperature overnight. The product was isolated by vacuum filtration under nitrogen flow, washed with IPA (30 ml), then dried in vacuum oven at 40° C. for to obtain FLV-diol-tert butyl ester. First crystallization-Syn:anti-98.9/0.61.

### Example 7

### Crystallization of Crude FLV-diol Ester From Acetone

Crude FLV-diol-t-Butyl ester (4.0 g) was dissolved in acetone (18.5 ml) at reflux temperature. After 45 minutes the clear solution was cooled to room temperature to obtain a massive precipitate. The suspension was diluted with Acetone (10 ml) and the product was isolated by vacuum filtration under nitrogen flow, washed with Acetone (4×10 ml) and dried in a vacuum oven at 50° C. for 24 hours to obtain FLV-diol-t-Butyl ester (1.7 g, 42%). First crystallization-Syn:anti-98.8/0.27; Second crystallization-Syn:anti-99.6/0.04.

### Example 8

### Crystallization of Crude FLV-diol Ester From Isobutylacetate

FDE-tBu (3 gr) (Syn:anti-98.6/0.88) was dissolved in Isobutylacetate (48 ml) by reflux. The solution was cooled to room temperature and stirred over night. The product was isolated by vacuum filtration, washed with isobutylacetate and dried in vacuum oven at 50° C. for 24 hours to obtain FDE-tBu (1.92 gr, 64% yield). First crystallization-Syn:anti-99.6/0.2.

### Example 9

### Crystallization of Crude FLV-diol Ester From IPA and MTBE

FDE-tBu (3 gr, syn:anti 98.6:0.88) was dissolved in IPA (15 ml) by reflux and MTBE (30 ml) was added. The solution was cooled to room temperature and stirred over night. The product was isolated by vacuum filtration, washed with a solution of MTBE:IPA 1:1 v:v (20 ml) and dried in vacuum oven at 40 deg for 24 hours to obtain FDE-tBu (1.5 gr, 51% yield). Syn:anti 99.6:0.20

### Example 10

### Reduction of TB -21 to TB-22 (tBu-Rosuvastatin) with B-OMe-9-BBN and NaBH₄

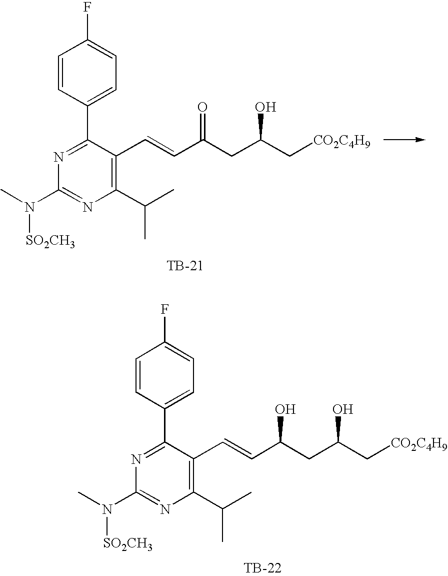

A 100 mL 3-necked flask equipped with a mechanical stirrer, rubber septum, and nitrogen bubbler was charged with TB-21 (1.0 g), tetrahydrofuran (47 mL) and methanol (13.5 mL). The mixture was stirred at room temperature until all TB-21 was dissolved. The reaction mixture was then cooled to −78° C. B-OMe-9-BBN (2.05 mL, 1 M in Hexanes) was added via a syringe at −78° C. and the solution was stirred for about 30 minutes. NaBH₄ (0.078 g) was added at −78° C. and the solution was stirred for about 3 hours. H₂O₂ (0.8 mL, 30% in water) was added at −78° C. The solution was then allowed to reach room temperature and the solution was evaporated to dryness. Ethyl acetate (5 mL) , water (5 mL) and NaCl (saturated, 3.5 mL) was added to the residue. The organic phase was separated and washed with saturated NaHCO₃ (4 mL), saturated Na₂SO₃ (4 mL), and saturated NaCl (4 mL). The combined organic layers were concentrated under reduced pressure to obtain a residue of the diol TB-22. (1.19 g, 92.0%). Diastereoisomer content is 0.37%.

### Example 11

### Crystallization of TBRE (TB22) From Toluene

TBRE (2 g, 0.23% diastereoisomers) was dissolved in Toluene (7 ml) by heating to approximately 60° C. The solution was then allowed to cool to room temperature, and was cooled afterwards in an ice bath to 0° C. The resulting mixture was stirred at this temperature overnight. The solid was then filtered under reduced pressure, washed, and dried at 50° C. under reduced pressure for 18 hrs to get 1.59 g of TBRE (0.08% diastereoisomers).

### Example 12

### Slurry TBRE in MeOH

TBRE (1 g, 1.1% of diastereoisomers) was suspended in MeOH (5 ml) while stirring at ambient temperature overnight. The solid was then filtered under reduced pressure, washed, and dried at 45° C. under atmospheric pressure for 18 hrs to obtain 0.60 g of TBRE (diastereoisomers 0.51%)

### Example 13

### Preparation of Rosuvastatin Calcium from Rosuvastatin Ester

A 1000 ml reactor equipped with a mechanical stirrer was charged with EtOH (100 mL) water (60 ml) t-Butyl-Rosuvastatin (20 g) and NaBH₄ (0.1 g). To this suspension, NaOH 47% 1.1 eq (3.5 g) was added dropwise at 25±5° C. and the mixture was stirred at 25±5° C. for two hours. The mixture was then filtered under reduced pressure with a Sinter to eliminate the active carbon present in the solution.

To this suspension water (140 ml) was added and the reaction mixture was acidified with HCl 0.1 M until PH 8-10. The mixture was then washed with Toluene (100 ml) and stirred at 25±5° C. for half an hour. The aqueous layer was isolated. To the aqueous phase active carbon was added and the suspension was stirred at 25±5° C. for 30 min. The mixture was filtered under reduced pressure with Sinter and Hyflo to eliminate the active carbon present in the solution. Thereafter the reaction mixture was concentrated under reduced pressure at 40° C. to half the solution volume.
Make-up of the solution was performed to 10 volumes of water versus TBRE. The solution was heated to 40-45 ° C. CaCl₂ (4.13 g) was added dropwise to this solution over 30-90 min at 38-45° C. The suspension was then cooled to 25±5° C., stirred at 25±5° C. for 1 hr, filtered and washed with water (4×20 ml) to get a powdery compound (17.3 g dry, 92%).

The resulting solution was placed in a flask and heated to 40° C. Solid CaCl₂ (0.25 g) was added portionwise to this solution while stirring. The resulting mixture was then cooled to 25±5° C., stirred at 25±5° C. for 1 hr, filtered and washed with water to get a powdery product, which was dried in vacuum at 50° C.

What is claimed is:

1. A process for preparing rosuvastatin diol ester comprising the steps of
a) combining B-Methoxy-9-BBN, an organic solvent, and a ketoester having the formula: 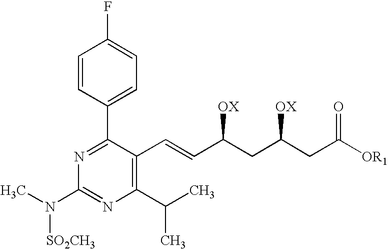
wherein R₁ is a straight or branched C₁ to C₄ alkyl group, and wherein at least one X forms a double bond to give a ketone, and at most one X is a hydrogen, to obtain a reaction mixture,
b) combining a source of hydride ions with the reaction mixture, and
c) maintaining the reaction mixture to obtain the rosuvastatin diol ester.

2. The process of claim 1, wherein the solvent is selected from the group consisting of: C₁ to C₄ alcohol, dipolar solvent, cyclic or acyclic C₂ to C₈ ether and a mixture thereof.

3. The process of claim 1, wherein the organic solvent is selected from a group consisting of methylene chloride, toluene, methyl t-butyl ether, di-ethyl ether, tetrahydrofuran, dioxane, methanol, ethanol, isopropanol, n-butanol.

4. The process of claim 1, wherein the solvent is a mixture of methanol and tetrahydrofuran.

5. The process of claim 1, further comprising cooling the reaction mixture of step
a) to a temperature of about −50° C. to about −80° C.;

6. The process of claim 5, wherein the solution is cooled to about −70° C. to about −80° C.

7. The process of claim 6, wherein the temperature is about −78° C.

8. The process of claim 1, further comprising recovering the rosuvastatin diol-ester.

9. The process of claim 1, wherein the source of the hydride ions is selected from the group consisting of: sodium borohydride, potassium borohydride and lithium borohydride.

10. The process of claim 1, wherein the source of the hydride ions is sodium borohydride.

11. The process of claim 1, wherein the reaction mixture is maintained for at least about 30 minutes.

12. The process of claim 1, further comprising quenching the reaction mixture.

13. The process of claim 12, wherein the quenching agent is selected from a group consisting of 3-chloroperbenzoic acid, ammonium chloride, aqueous solution of HCl, acetic acid, oxone, sodium hypochlorite, dimethyl disulfide, diethanolamine, acetone and hydroxylamine-O-sulfonic acid.

14. The process of claim 13, wherein the quenching agent is hydrogen peroxide.

15. The process of claim 1, wherein the ketoester is an alpha ketoester.

16. The process of claim 1, further comprising crystallizing or slurrying the diol ester from an organic solvent or a mixture of water and an organic solvent.

17. The process of claim 16 wherein crystallizing the diol ester comprises:
a) preparing a heated solution of the diol ester in an organic solvent, mixtures of organic solvents, and mixtures of water and organic solvents;
b) cooling the solution to crystallize the diol ester; and
c) recovering the crystalline diol ester.

18. The process of claim 17, wherein the organic solvent is selected from a group consisting of C₁-C₄ alcohols, C₃-C₈ esters, C₃-C₈ ketones, C₃-C₈ ethers, PGME (propylene glycol monomethyl ether), acetonitrile, and mixtures thereof.

19. The process of claim 17, wherein the organic solvent or mixture thereof with water is selected from the group consisting of methanol, PGME, acetonitrile:water, acetone:water, acetone:MTBE (methyl tert-butyl ether), methanol:water, ethanol:water, ethanol:MTBE, acetonitrile: MTBE, methanol:MTBE, MEK (methyl ethyl ketone):MTBE and toluene.

20. The process of claim 17, wherein the solvent is PGME.

21. The process of claim 17, wherein the solvent is a mixture of acetonitrile and water.

22. The process of claim 17, wherein the solvent is a mixture of acetone and water.

23. The process of claim 17, wherein the solvent is a mixture of acetone and MTBE.

24. The process of claim 17, wherein the solvent is a mixture of methanol and water.

25. The process of claim 17, wherein the solvent is a mixture of ethanol and water.

26. The process of claim 17, wherein the solvent is a mixture of ethanol and MTBE.

27. The process of claim 17, wherein the solvent is a mixture of methanol and MTBE.

28. The process of claim 17, wherein the solvent is a mixture of MEK and MTBE.

29. The process of claim 17, wherein the solvent is toluene.

30. The process of claim 17, wherein heated solution is at a temperature above about 50° C.

31. The process of claim 17, wherein cooling in step b) is to a temperature of about 40° C. to about 0° C.

32. The process of claim 17, wherein cooling in step b) is to a temperature of about 30° C. to about 0° C.

33. The process of claim 17, wherein cooling in step b) is to a temperature of about 5° C. to about 0° C.

34. A process for preparing rosuvastatin further comprising converting the diol ester of claim 1 to rosuvastatin or a pharmaceutically acceptable salt thereof.

35. A pharmaceutical composition comprising rosuvastatin or a pharmaceutically salt thereof prepared according to claim 34 and at least one pharmaceutically acceptable excipient.

36. A method of inhibiting the 3-hydroxy-3-methyl-glutaryl-coenzyme A (“HMG-CoA”) reductase enzyme in a mammal in need thereof comprising administering the pharmaceutical composition of claim 35 to the mammal.

37. A process for preparing rosuvastatin from a rosuvastatin diol-ester having the formula: 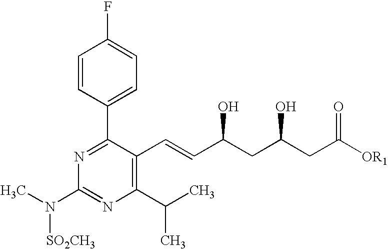
wherein R₁ is a straight or branched C₁ to C₄ alkyl group; comprising the steps of
a) combining a ketoester of rosuvastatin having the formula: 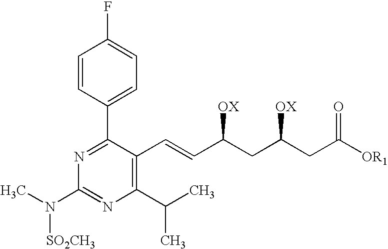
with a solvent to form a solution, wherein R₁ is a straight or branched C₁ to C₄ alkyl group, and wherein at least one X forms a double bond to give a ketone, and at most one X is a hydrogen;
b) cooling the solution to a temperature of about −50° C. to about −80° C.;
c) combining B-Methoxy-9-BBN with the solution to obtain a reaction mixture, and maintaining the reaction mixture for at least about 30 minutes;
d) combining a source of hydride ions with the reaction mixture, and maintaining the reaction mixture for an additional period of at least about 2 hours;
e) quenching the reaction mixture;
f) recovering the rosuvastatin diol-ester; and
g) converting the rosuvastatin diol-ester to rosuvastatin or a pharmaceutically acceptable salt of rosuvastatin.

38. The process of claim 37, wherein the pharmaceutically acceptable salt is calcium salt or sodium salt.

39. A process for preparing rosuvastatin from a rosuvastatin ketoester having the formula: 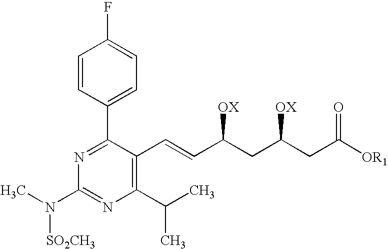
wherein R₁ is a straight or branched C₁ to C₄ alkyl group, and wherein at least one X forms a double bond to give a ketone, and at most one X is a hydrogen, comprising the steps of
a) combining the ketoester of rosuvastatin with a solvent to form a solution;
b) cooling the solution to a temperature of about −50° C. to about −80° C.;
c) combining B-Methoxy-9-BBN with the solution to obtain a reaction mixture and maintaining the reaction mixture for at least about 30 minutes;
d) combining a source of the hydride ions to the reaction mixture and maintaining the reaction mixture for an additional period of at least about 2 hours to obtain rosuvastatin diol ester;
e) quenching the reaction mixture;
f) combining the rosuvastatin diol ester with NaOH or Ca(OH)₂ and a solvent or a mixture of solvent and water; and
g) recovering the rosuvastatin free acid, lactone or a pharmaceutically acceptable salt thereof.
